(19) United States
(12) Patent Application Publication
(10) Pub. No.: US 2017/0273568 A1
(43) Pub. Date: Sep. 28, 2017

(54) PHOTOACOUSTIC APPARATUS AND PROCESSING METHOD FOR PHOTOACOUSTIC APPARATUS

(71) Applicant: CANON KABUSHIKI KAISHA, Tokyo (JP)

(72) Inventor: Takuro Miyasato, Tokyo (JP)

(21) Appl. No.: 15/510,743

(22) PCT Filed: Oct. 7, 2015
(86) PCT No.: PCT/JP2015/005098

§ 371 Date: Mar. 13, 2017

(30) Foreign Application Priority Data
Oct. 10, 2014 (JP) 2014-209193

(51) Int. Cl. A61B 5/00; G01N 29/24; G01N 29/46; G01N 29/06

(52) U.S. Cl. CPC A61B 5/0095; G01N 29/0672; G01N 29/2418; G01N 29/46; A61B 5/4312; A61B 5/708; G01N 2291/015; G01N 2291/02466

(57) ABSTRACT

A photoacoustic apparatus comprises a light source; an acoustic wave receiver receives an acoustic wave and converts into an electric signal; a first acquisition unit acquires a first absorption coefficient distribution inside the object using a first method; a second acquisition unit acquires a second absorption coefficient distribution inside the object using a second method; a third acquisition unit calculates the distribution of functional information on the interior of the object; and an image generation unit generates an image by masking the distribution of the functional information based on the second absorption coefficient distribution, wherein the second method is a method that can implement higher visibility than the first method when the absorption coefficient distribution is imaged.

### TECHNICAL FIELD

The present invention relates to a photoacoustic apparatus that acquires information on the interior of an object.

### BACKGROUND ART

Currently in medical fields, research on techniques to image form information and physiological information (functional information) on the interior of an object are ongoing. As one such technique, photoacoustic tomography (PAT) has been proposed in recent years.

If light, such as pulsed laser light, is irradiated to a living body (object), an acoustic wave (typically an ultrasonic wave) is generated when the light is absorbed by a biological tissue inside the object. This phenomenon is called the “photoacoustic effect”, and an acoustic wave generated by the photoacoustic effect is called the “photoacoustic wave”. Since each tissue constituting the object has different light energy absorptivity, the sound pressure of the photoacoustic wave to be generated from each tissue is also different. In PAT, the generated photoacoustic wave is received by a probe and the received signal is mathematically analyzed, whereby the optical characteristics inside the object, particularly the distribution of light absorption coefficients, can be imaged.

Furthermore, based on the acquired light absorption coefficient distribution, the percentage of oxyhemoglobin content with respect to all hemoglobin in blood, that is oxygen saturation, can be determined. Since oxygen saturation becomes an index to discern whether a tumor is benign or malignant, photoacoustic tomography is expected as an efficient means to discover a malignant tumor.

By using these techniques together, both the form information on the interior of the object (e.g. vascular structure) and the functional information (e.g. oxygen saturation) can be acquired.

### CITATION LIST

### Patent Literature

[Patent Literature 1] Japanese Patent Application Laid-Open No. 2011-217767
[Patent Literature 2] Japanese Patent Application Laid-Open No. 2013-233414
[Patent Literature 3] Japanese Patent Application Laid-Open No. 2013-053863

### SUMMARY OF INVENTION

### Technical Problem

To image form information, such as the vascular structure inside an object, it is necessary to clearly display information on the locations of the blood vessels to the operator of the apparatus. Therefore, processing to improve visibility of the image is normally executed.

As an example of this technique, Patent Literature 1 discloses processing to delete an artifact generated in the image. Patent Literature 2 discloses a method for highlighting a true signal portion by restoring signals based on a model. And Patent Literature 3 discloses a method for improving the visibility of an image by blind deconvolution.

On the other hand, in the case of imaging functional information, such as oxygen saturation, it is more critical to improve the accuracy of the values than the visibility of the image, since the values indicate whether a tumor is benign or malignant.

The oxygen saturation is measured a plurality of times using pulsed light having different wavelengths, and is calculated by comparing calculated light absorption coefficients. In other words, to accurately calculate the oxygen saturation, the ratio of the absorption coefficients, based on [the specific oxygen saturation] that is calculated, must be accurate. However, if the above mentioned processing to improve the visibility of the image is executed, the ratio of the absorption coefficients among the wavelengths will change, and the accuracy of the oxygen saturation will drop.

On the other hand, as a technique to improve the accuracy of the oxygen saturation, a method of blurring an image, a method of narrowing the view angle or the like is known, but if such a method is executed, the imaging accuracy of a vascular image will drop.

With the foregoing problems of the prior art in view, it is an object of the present invention to implement measuring accuracy for both the structural information and functional information on the interior of the object in the photoacoustic apparatus.

### Solution to Problem

The present invention in its one aspect provides a photoacoustic apparatus, comprises a light source configured to irradiate a plurality of pulsed light having different wavelengths to an object; an acoustic wave receiver configured to receive an acoustic wave generated from the object, to which the pulsed light have been irradiated, and convert the acoustic wave into an electric signal; a first information acquisition unit configured to acquire a first absorption coefficient distribution inside the object, based on the electric signal, using a first calculation method; a second information acquisition unit configured to acquire a second absorption coefficient distribution inside the object, based on the electric signal, using a second calculation method; a third information acquisition unit configured to calculate the distribution of functional information on the interior of the object based on the plurality of first absorption coefficient distributions acquired by irradiating the plurality of pulsed light having different wavelengths respectively; and an image generation unit configured to generate an image by masking the distribution of the functional information based on the second absorption coefficient distribution, wherein the second calculation method is a method that can implement higher visibility than the first calculation method when the absorption coefficient distribution is imaged.

The present invention in its another aspect provides a processing method for a photoacoustic apparatus having a light source configured to irradiate a plurality of pulsed light having different wavelengths to an object, and an acoustic wave receiver configured to receive an acoustic wave generated from the object, to which the pulsed light have been irradiated, and convert the acoustic wave into an electric signal, the method comprises a first information acquisition step of acquiring a first absorption coefficient distribution inside the object, based on the electric signal, using a first calculation method; a second information acquisition step of acquiring a second absorption coefficient distribution inside the object, based on the electric signal, using a second calculation method; a third information acquisition step of calculating a distribution of functional information on the interior of the object based on the plurality of first absorption coefficient distributions acquired by irradiating the plurality of pulsed light having different wavelengths respectively; and an image generation step of generating an image by masking the distribution of the functional information based on the second absorption coefficient distribution, wherein the second calculation method is a method that can implement higher visibility than the first calculation method when the absorption coefficient distribution is imaged.

### Advantageous Effects of Invention

According to the present invention, measurement accuracy can be implemented for both the structural information and functional information on the interior of the object in the photoacoustic apparatus.

Further features of the present invention will become apparent from the following description of exemplary embodiments with reference to the attached drawings.

### [BRIEF DESCRIPTION OF DRAWINGS]

FIG. 1 is a system block diagram of a photoacoustic measurement apparatus according to Embodiment 1;

FIG. 2 is a processing flow chart of the photoacoustic measurement apparatus according to Embodiment 1;

FIG. 3 is a system block diagram of a photoacoustic measurement apparatus according to Embodiment 2;

FIGS. 4A to FIG. 4F are diagrams depicting examples of an absorption coefficient distribution according to Example 1;

FIGS. 5A to FIG. 5F are diagrams depicting examples of oxygen saturation distribution according to Example 1;

FIGS. 6A to FIG. 6D are diagrams depicting examples of absorption coefficient distribution according to Example 2; and

FIGS. 7A to FIG. 7D are diagrams depicting examples of oxygen saturation distribution according to Example 2.

### DESCRIPTION OF EMBODIMENTS

Embodiments of the present invention will now be described with reference to the drawings. The numeric values, materials, shapes, positions or the like used for the description of the embodiments should be appropriately changed depending on the configuration and various conditions of the apparatus to which the present invention is applied, and are not intended to limit the scope of the invention.

The configuration of the apparatus and overview of the processing will be described in Embodiments 1 and 2 first, and, based on the description, concrete content of the processing will be described in Examples 1 to 5.

### Embodiment 1

A photoacoustic apparatus according to Embodiment 1 is an apparatus to visualize (to image) the structural information and functional information on the interior of an object by irradiating a pulsed light into the object, and receiving and analyzing the photoacoustic wave that is generated inside the object by the pulsed light. The structural information is the initial sound pressure distribution, the light absorption energy density distribution, or the object information related to the absorption coefficient distribution derived from the initial sound pressure distribution and light absorption energy density distribution, and is primarily the light absorber structure information on the interior of the object, particularly the vascular structure information. The functional information is the spectral information calculated using the photoacoustic signals and spectrum information acquired with a plurality of wavelengths. The functional information is primarily the information on biological functions, such as the substance concentration inside the object, particularly oxygen concentration in the blood inside the blood vessels, or the concentration of fat, collagen, hemoglobin or the like.

### <System Configuration>

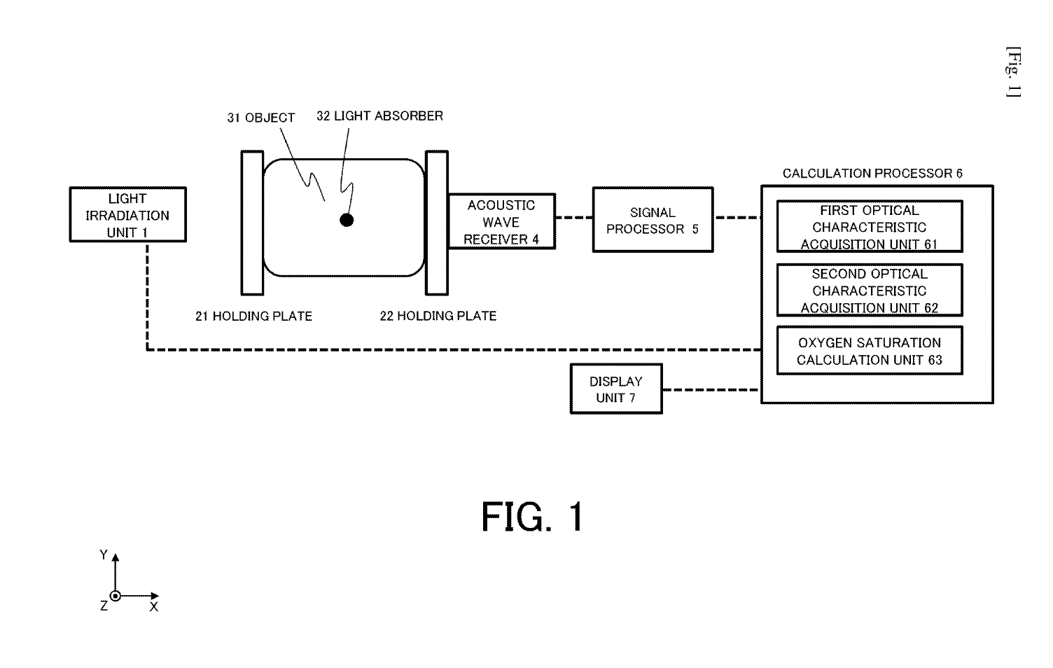

A configuration of a photoacoustic measurement apparatus according to this embodiment will be described with reference to FIG. 1. The photoacoustic measurement apparatus according to this embodiment has a light irradiation unit 1, holding plates 21 and 22, an acoustic wave receiver 4, a signal processor 5, a calculation processor 6, and a display unit 7. The calculation processor 6 includes a first optical characteristic acquisition unit 61, a second optical characteristic acquisition unit 62, and an oxygen saturation calculation unit 63.

An overview of the measurement method will now be described by describing each unit constituting the photoacoustic measurement apparatus according to this embodiment.

### <<Light Irradiation Unit 1>>

The light irradiation unit 1 is a unit configurated to generate a pulsed light and irradiate the pulsed light to an object, and is constituted by a light source and an irradiation optical system.

The light source is preferably a laser light source to acquire high output, but may be a light emitting diode, a flash lamp or the like instead of a laser. In the case of using laser as the light source, various lasers can be used, including a solid state laser, a gas laser, a dye laser and a semiconductor laser.

Ideally an Nd:YAG excited OPO laser, a dye laser, a Ti:sa laser or an alexandrite laser should be used since output is high and wavelength can be continuously changed. A plurality of single wavelength lasers having different wavelengths may be used.

The wavelength of the pulsed light is preferably a specific wavelength absorbed by a specific component out of the components constituting the object, and is a wavelength by which the light is propagated into the object. In concrete terms, if the object is a living body, the wavelength is preferably 500 nm to 1200 nm.

To effectively generate a photoacoustic wave, the light must be irradiated in a sufficiently short time according to the thermal characteristics of the object. If the object is a living body, the pulse width of the pulsed light generated from the light source is preferably about 5 to 50 nanoseconds. The pulsed light generated from the light source is hereafter called the “irradiation light”.

The light source need not always be a part of the photoacoustic measurement apparatus according to this embodiment, but may be connected externally.

The irradiation light emitted from the light source is irradiated toward an object via an irradiation optical system. The irradiation optical system is constituted by optical members, such as a mirror to reflect light, a lens to expand light, and a diffusion plate to diffuse light. A combination of an optical fiber, bundled optical fiber, lens barrel and mirror, for example, may be used. The irradiation optical system can be any system if the irradiation light emitted from the light source can be irradiated in a desired shape toward the object. It is preferable to expand the light to a certain area rather than to collect the light by a lens, since the diagnostic region in the object can be expanded.

A scanning mechanism to move the irradiation optical system on the surface of the object may be disposed. If a pulsed light having a desired shape can be irradiated directly from the light source, the irradiation optical system is not always required.

### <<Holding Plates 21 and 22>>

The holding plates 21 and 22 are units that hold an object. In concrete terms, one or both of the plate type holding members move(s) in the X-axis direction to compress and hold the object.

The measurement light emitted from the light irradiation unit 1 is irradiated onto the surface of the object via the holding plate 21, hence the holding plate 21 is preferably formed of a material of which transmittance of the measurement light is high and decay rate is low. Typically glass, polymethylpentene, polycarbonate, acryl or the like is preferable.

The acoustic wave generated inside the object enters the acoustic wave receiver 4 via the holding plate 22. Therefore the holding plate 22 is preferably formed of a material which does not reflect the acoustic wave on the boundary surface with the object, and which can easily transmit an acoustic wave. In concrete terms, a material of which difference of acoustic impedance from the object is small is preferable. An example of such a material is a resin material including polymethylpentene.

### <<Object 31 and Light Absorber 32>>

The object 31 and light absorber 32 will be described here, although neither constitute the apparatus. The photoacoustic measurement apparatus according to this embodiment is primarily used for angiography, the diagnosis of malignant tumors and vascular diseases of humans and animals, and the follow-up observation of chemotherapy or the like. Therefore the object 31 is assumed to be a living body part, such as a breast, finger, limb and other diagnostic target segments of humans and animals. If the object is a small animal, then not only a specific segment, but an entire body as well may become the target.

A light absorber 32 existing inside the object 31 is a segment of which absorption coefficient, with respect to light, is relatively high within the object. For example, if the measurement target is a human body, the light absorber 32 could be oxyhemoglobin, deoxyhemoglobin, blood vessels including red blood cells, and malignant tumors containing neovessels. Melanin or the like on the surface of the object 31 also could be a light absorber 32. The light absorber 32 may be such a dye as methylene blue (MB) and indocyanine green (ICG), fine gold particles, and a substance generated by integrating or chemically modifying these substances.

### <<Acoustic Wave Receiver 4>>

The acoustic wave receiver 4 is a unit to receive an acoustic wave generated inside the object, and convert the acoustic wave into an electric signal. The acoustic wave receiver is also called the “acoustic wave detector” or the “transducer”. The acoustic wave in the present invention is typically an ultrasonic wave, and includes an elastic wave called the “sound wave”, the “ultrasonic wave”, the “photoacoustic wave” and the “light induced ultrasonic wave”.

The acoustic wave generated from a living body is a 100 KHz to 100 MHz ultrasonic wave, hence an acoustic element that can receive this frequency band is used for the acoustic wave receiver 4. In concrete terms, a transducer using the piezoelectric phenomenon, a transducer using the resonance of light, a transducer using the change of capacitance or the like can be used. The acoustic wave receiver 4 preferably has high sensitivity and a wide frequency band.

The acoustic wave receiver 4 may have a plurality of acoustic wave receiving elements which are arrayed one-dimensionally or two-dimensionally. By receiving the acoustic wave simultaneously at a plurality of positions, the measurement time can be decreased and the influence of vibration of the object or the like can be reduced. The acoustic wave receiver 4 may be configured to be mechanically scannable using the scanning mechanism. The acoustic wave receiver 4 may include an acoustic lens.

### <<Signal Processor 5>>

The signal processor 5 is a unit to amplify an acquired electric signal (hereafter called the “photoacoustic signal”) and convert the electric signal into a digital signal. Typically the signal processor 5 is constituted by an amplifier, an A/D converter, an FPGA (Field Programmable Gate Array) chip and the like. If a plurality of signals is acquired from the acoustic wave receiver 4, it is preferable that [the signal processor 5] can process a plurality of signals simultaneously. The photoacoustic signal in this description is a concept that includes both the analog electric signal acquired by the acoustic wave receiver 4 and the digital signal converted by the signal processing mechanism.

The photoacoustic signals received at a same position in the object may be integrated into one signal. The integration method may be to add the signals or average the signals. Each signal may be weighted and added.

### <<Calculation Processor 6>>

The calculation processor 6 is a unit to control processing to acquire the information on the interior of the object based on the intensity of the light irradiated to the object, the timing to irradiate light, the timing to receive the acoustic wave, and the received acoustic wave. The calculation processor 6 is also a unit to acquire the information related to the optical characteristics inside the object by calculating the light quantity distribution and reconstructing images based on the acquired photoacoustic signals. The calculation processor 6 corresponds to the first information acquisition unit, the second information acquisition unit, and the image generation unit of the present invention.

The calculation processor 6 is typically a workstation, and executes the above mentioned processing using software which is stored in advance.

In this embodiment, the calculation processor 6 includes the first optical characteristic acquisition unit 61, the second optical characteristic acquisition unit 62, and the oxygen saturation calculation unit 63, and these units are installed as software.

In this embodiment, the calculation processor 6 is a workstation, but the calculation processor 6 may be a set of a plurality of hardware. In this case the total of each hardware is called the “calculation processor 6”.

### <<Display Unit 7>>

The display unit 7 is an apparatus to display an image of the information generated by the calculation processor 6, and is typically a liquid crystal display, but may be another type of display, such as a plasma display, an organic EL display and an FED.

The display unit 7 need not always be a part of the photoacoustic measurement apparatus according to this embodiment, but may be connected externally.

### <<Object Measurement Method>>

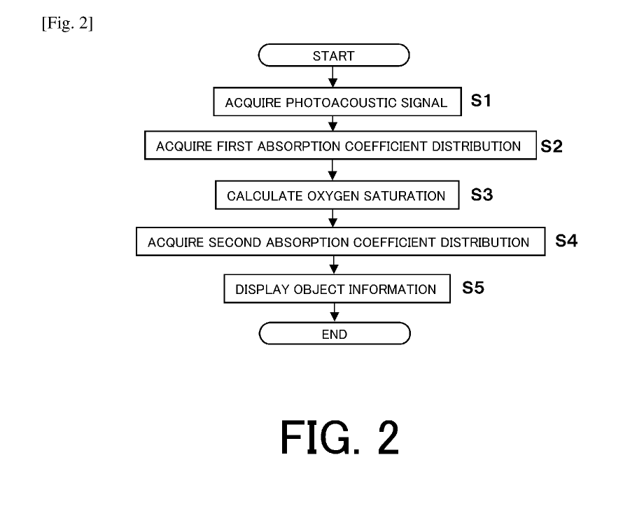

Now a method of measuring a living body (object) by the photoacoustic measurement apparatus according to this embodiment will be described with reference to the processing flow chart in FIG. 2.

First a pulsed light having a specific wavelength is irradiated from the light irradiation unit 1 into the object. The pulsed light is irradiated after being guided to the surface of the object while being processed to have a desired shape by the irradiation optical system constituted by a lens, a mirror, an optical fiber, a diffusion plate and the like. When a part of the energy of the light propagated inside the object 31 is absorbed by such a light absorber 32 as blood vessels, a photoacoustic wave (typically an ultrasonic wave) is generated by the thermal expansion of the light absorber 32.

The generated acoustic wave propagates inside the object, is received by the acoustic wave receiver 4 via the holding plate 22, and is converted into a photoacoustic signal (S1).

The photoacoustic measurement apparatus according to this embodiment executes this measurement for a plurality of times, with changing the wavelength of the pulsed light each time. The photoacoustic signal corresponding to each wavelength is temporarily stored by the calculation processor 6.

Before advancing to the next step, a method of generating an image expressing object information based on the stored photoacoustic signals will be described. First a method of calculating a distribution of the absorption coefficients inside the object will be described. The initial sound pressure P₀ of the acoustic wave generated by the light absorber in the object is given by Expression 1.

P₀=Γ·μₐ·Φ (Expression 1)

Here Γ is a Grueneisen coefficient determined by dividing the product of the volume expansion coefficient β and a square of the sound velocity c by the specific heat at constant pressure Cₚ. It is known that the value of Γ is almost constant if the object is determined. μₐ is a light absorption coefficient of an absorber, and Φ is a light quantity in a local region (light quantity irradiated into the absorber, and is also called the “light fluence”).

If the time-based change of the sound pressure P, which is a magnitude of the acoustic wave propagated inside the object, is measured, the initial sound pressure distribution can be calculated from the measurement result. Further, the absorption coefficient distribution μₐ can be calculated by dividing the calculated initial sound pressure distribution by the Grueneisen coefficient Γ and the light quantity distribution Φ inside the object. One method to determine the initial sound pressure from the photoacoustic signal is the universal back projection method (hereafter called the “UBP method”). Detailed description of this method is omitted here since [the UBP method] is publically known. In the following description, it is assumed that P(λ₁) denotes the initial sound pressure of the acoustic wave, which is generated corresponding to the light with the wavelength λ₁, and P(λ₂) denotes the initial sound pressure of the acoustic wave which is generated corresponding to the light with the wavelength λ₂.

Then a method of calculating the distribution of the oxygen saturation inside the object will be described. The oxygen saturation, which is spectral information, must be calculated using the absorption coefficient distributions acquired using different wavelengths. The oxygen saturation StO inside the object is given by Expression 2.

StO=[(E_HbR(λ₂)−{A(λ₂)/A(λ₁)}×E_HbR(λ₁))]/[{E_HbR(λ₂)−E_HbO(λ₂)}−{A(λ₂)/A(λ₁)}×{E_HbR(λ₁)−E_HbO(λ₁)}]×100 (Expression 2)

Here StO denotes oxygen saturation, and E_HbR (λ₁) and E_HbR (λ₂) denote the absorption coefficients per 1 mol/liter of deoxyhemoglobin with respect to wavelength λ₁ and wavelength λ₂ respectively. E_HbO (λ₁) and E_HbO (λ₂) denote the absorption coefficients per 1 mol/liter of oxyhemoglobin with respect to wavelength λ₁ and wavelength λ₂ respectively. λ(λ₁) denotes an absorption coefficient inside the object corresponding to wavelength λ₁, and λ(λ₂) denotes an absorption coefficient inside the object corresponding to wavelength λ₂.

In this embodiment, the calculation processor 6 determines the absorption coefficient distribution and the oxygen saturation distribution inside the object by the above mentioned method, generates a corresponding image, and displays the image to the operator via the display unit 7.

As mentioned above, high visibility is demanded when the absorption coefficient distribution is displayed, and good quantitativity is demanded when the oxygen saturation distribution is displayed. However, a conventional method cannot satisfy these two demands simultaneously. For example, if processing to improve the visibility of the vascular structure is executed, the ratio of the absorption coefficients between wavelengths becomes incorrect, and accuracy of oxygen saturation drops, and if processing to increase the accuracy of oxygen saturation is executed, visibility of vascular structure drops.

Therefore in this embodiment, the calculation processor 6 calculates “the absorption coefficient distribution to display the vascular structure” and “the absorption coefficient distribution to calculate the oxygen saturation” independently using different methods, and generates respective images.

In concrete terms, the first optical characteristic acquisition unit 61 calculates the absorption coefficient distribution by a first calculation method based on the stored photoacoustic signal (S2). The first calculation method is a method whereby the radio of the absorption coefficients between different wavelengths become close to a true value. In other words, this is a calculation method suitable for calculating the oxygen saturation. The absorption coefficient distribution determined like this is called the “first absorption coefficient distribution”.

Then the oxygen saturation calculation unit 63 calculates the oxygen saturation distribution based on the first absorption coefficient distribution (S3).

The second optical characteristic acquisition unit 62 calculates the absorption coefficient distribution by a second calculation method based on the stored photoacoustic signal (S4). The second calculation method is a method whereby visibility increases when the calculated absorption coefficient distribution is imaged. In other words, this is a calculation method suitable for displaying the vascular structure. The absorption coefficient distribution determined like this is called the “second absorption coefficient distribution”.

The first calculation method and the second calculation method will be described. The first calculation method and the second calculation method are both a series of processing to calculate the absorption coefficient based on a photoacoustic signal, but are different in terms of the included processing and the image reconstruction method.

The first calculation method includes processing to make the ratio of the absorption coefficients between different wavelengths at a certain pixel or voxel existing inside the blood vessels to be closer to the actual ratio of the absorption coefficients. For example, the influence from different blood vessels (reconstruction artifact) can be decreased by decreasing the view angle of the probe used for reconstruction (hereafter called the “reconstruction view angle”). Other processing may be included only if a ratio of absorption coefficients between different wavelengths can be made closer to the actual ratio of the absorption coefficients. Concrete examples will be shown in the later mentioned examples.

The first optical characteristic acquisition unit 61 performs processing for the initial sound pressures P(λ₁) and P(λ₂) using the first calculation method, and acquires the first absorption coefficient distributions B(λ₁ and B(λ₂).

The second calculation method includes processing to improve visibility when the absorption coefficients are imaged. For example, the resolution and contrast of the blood vessels can be enhanced by performing processing to delete artifact by a plane wave, or processing to restore an image or a signal, whereby visibility when the absorption coefficients are imaged can be improved. The visibility when the absorption coefficients are imaged can also be improved by increasing the view angle of a probe used for reconstruction (hereafter called the “reconstruction view angle”). Other processing may be included only if visibilty when the absorption coefficients are imaged can be improved. Concrete examples will be shown in the later mentioned examples.

The second optical characteristic acquisition unit 62 performs processing for the initial sound pressures P(λ₁) and P(λ₂) using the second calculation method, and acquires the second absorption coefficient distributions C(λ₁ and C(λ₂).

The photoacoustic measurement apparatus according to this embodiment generates an image expressing the vascular structure and the oxygen saturation based on the first absorption coefficient distribution and the second absorption coefficient distribution (S5). In concrete terms, an image where the oxygen saturation, which is calculated using the first absorption coefficient distribution, is plotted for hue, and the absorption coefficients expressed by the second absorption coefficient distribution is plotted for lightness, is generated. Then one image can express both the oxygen saturation and the vascular structure. The generated image is displayed to the operator of the apparatus via the display unit 7.

In this embodiment, an image is generated plotting the oxygen saturation for hue and the second absorption coefficient distribution for lightness, but other methods may be used if the distribution of the functional information (oxygen saturation) can be masked by the second absorption coefficient distribution. For example, the oxygen saturation distribution may be masked using the data generated by binarizing the second absorption coefficient distribution into a transmitted portion and a non-transmitted portion.

### Embodiment 2

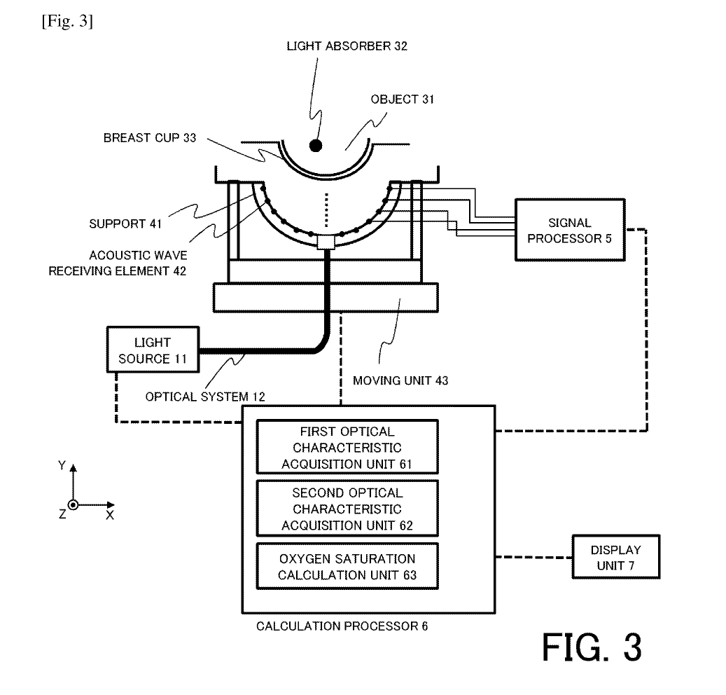

Embodiment 2 is a photoacoustic measurement apparatus configured to perform measurement on a human breast using a hemispherical acoustic wave receiver. FIG. 3 is a system block diagram of the photoacoustic measurement apparatus according to Embodiment 2.

The photoacoustic measurement apparatus according to Embodiment 2 uses a light source 11 and an optical system 12 as the light irradiation unit. The light source 11 is a laser light source that can emit a 10 nanoseconds or shorter short pulsed light at two wavelengths: 756 nm and 797 nm.

The radius of the pulsed light emitted from the light source 11 is expanded to a certain size using the optical system 12 constituted by such optical members as a mirror and beam expander, then the pulsed light is irradiated to the object.

The photoacoustic measurement apparatus according to Embodiment 2, as an acoustic wave receiver, uses a hemispherical support 41 and a plurality of acoustic wave receiving elements 42 disposed on the support 41. The support 41 has a hemispheric shape of which radius is 127 mm, and the plurality of acoustic wave receiving elements 42 are disposed so as to face the center of the curvature of the hemisphere. The acoustic wave receiving element 42 is a cMUT element, of which size is 2 mm and band is 2 MHz (100%).

The pulsed light emitted from the optical system 12 is irradiated from the base of the support 41 toward the object in the positive Y axis direction.

The support 41 is configured to be rotated by a moving unit 43 on the X-Z plane with the Y axis at the center, as indicated by the dotted line in FIG. 3. In other words, by rotating the support 41, the plurality of acoustic wave receiving elements disposed on the support 41 can be moved with respect to the object. By this configuration, the acoustic wave can be received at a plurality of positions with respect to the object, and measurement accuracy can be improved.

The object 31 (breast) is held by a breast cup 33 made of polyethylene. In other words, the pulsed light emitted from the light source 11 is irradiated onto the surface of the breast via the breast cup 33. The irradiation light irradiated via the breast cup 33 is diffused within the breast, and is absorbed by the light absorber 32 inside the breast. The acoustic wave generated from the light absorber 32 is received by the acoustic wave receiving elements 42 disposed on the support 41.

A signal processor 5 is a unit to simultaneously receive signals outputted from a plurality of acoustic wave receiving elements 42, perform amplification and digital conversion on the signals, and transfer the processed signals to a calculation processor 6.

The calculation processor 6 is a unit to control processing to acquire information on the interior of the object based on the intensity of the light irradiated to the object, the irradiation timing of the light, the reception timing of the acoustic wave, the position of the moving unit, and the received acoustic wave. The calculation processor 6 is also a unit to process a photoacoustic signal outputted by the signal processor 5, and generate an image.

The calculation processor 6 includes a first optical characteristic acquisition unit 61, a second optical characteristic acquisition unit 62, and an oxygen saturation calculation unit 63, just like Embodiment 1. These units are installed as software which run on a workstation, just like Embodiment 1.

In Embodiment 2, a photoacoustic signal acquired by the signal processor 5 is stored in a memory in the calculation processor 6, and is processed by software. The subsequent processing is the same as Embodiment 1.

Now the effect of photoacoustic measurement apparatuses according to the embodiments will be described using concrete examples. Examples 1, 2, 4 and 5 are examples of the photoacoustic measurement apparatus according to Embodiment 2, and Example 3 is an example of the photoacoustic measurement apparatus according to Embodiment 1. In each case, the processing executed by the first optical characteristic acquisition unit 61 and the processing executed by the second optical characteristic acquisition unit 62 are different.

### EXAMPLE 1

In Example 1, the first optical characteristic acquisition unit 61 calculates the initial sound pressure distribution by the UBP method assuming that the reconstruction view angle is 10°, and then calculates the absorption coefficient distribution based on this initial sound pressure distribution. This method is the first calculation method in Example 1.

On the other hand, the second optical characteristic acquisition unit 62 calculates the initial sound pressure distribution by the UBP method assuming that the reconstruction view angle is 15°, and then calculates the absorption coefficient distribution based on this initial sound pressure distribution. This method is the second calculation method in Example 1.

The reconstruction view angle is a maximum angle with respect to the maximum sensitivity direction of an element which becomes a processing target when a signal, received by the element, is back-projected to the reconstruction region.

The effect when the first calculation method and the second calculation method are designed in this way will be described.

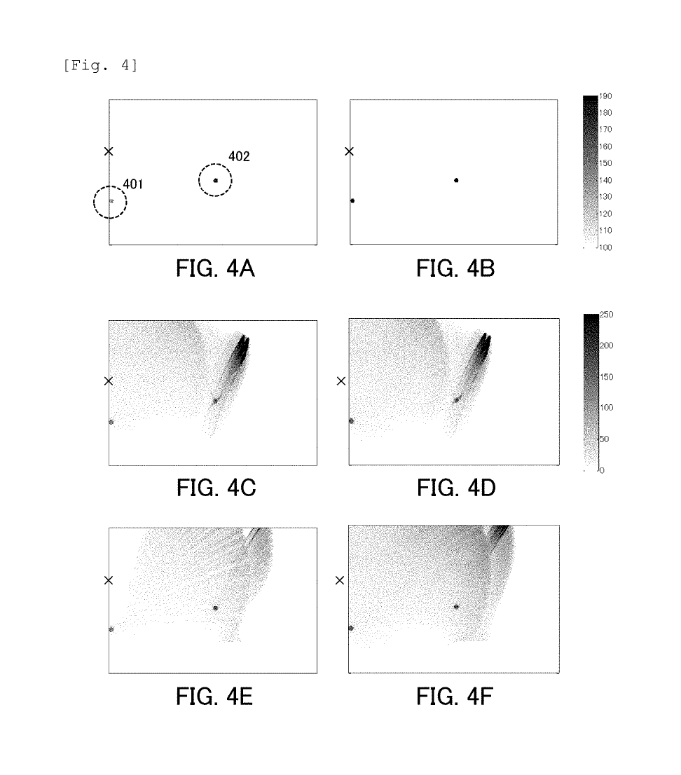

FIG. 4A is an absorption coefficient distribution acquired by irradiating the pulsed light having a 756 nm wavelength to the object, and FIG. 4B is an absorption coefficient distribution acquired by irradiating the pulsed light having a 797 nm wavelength to the object. Two light absorbers exist inside the object, and the absorber 401 on the left is an absorber of which oxygen saturation is 96%, which corresponds to an artery. The absorption coefficient of this absorber is 0.138/mm at a 756 nm wavelength, and is 0.189/mm at a 797 nm wavelength.

The absorber 402 on the right is an absorber of which oxygen saturation is 76%, which corresponds to a vein. The absorption coefficient of this absorber is 0.185/mm at a 756 nm wavelength, and is 0.189/mm at a 797 nm wavelength.

“X” in the image indicates the center point of the curvature of the support, and the pulsed light is irradiated from the lower side of each drawing. The size of each drawing shown in FIG. 4A to FIG. 4F is 50 mm (X direction)×30 mm (Y direction). The scale on the right side of each drawing indicates a dynamic range (/m).

For the absorption coefficient of a region other than the absorbers, an average optical coefficient of a human breast was used. In concrete terms, the absorption coefficient at a 756 nm wavelength is assumed to be 0.00265/mm, and the absorption coefficient at a 797 nm wavelength is assumed to be 0.00207/mm. The scattering coefficient at a 756 nm wavelength is assumed to be 0.817/mm, and the scattering coefficient at a 797 nm wavelength is 0.790/mm.

FIG. 4C and FIG. 4D are absorption coefficient distributions calculated assuming that the reconstruction view angle is 10°. FIG. 4C corresponds to the 756 nm wavelength, and FIG. 4D corresponds to a 797 nm wavelength. FIG. 4E and FIG. 4F are absorption coefficient distributions calculated assuming that the reconstruction view angle is 15°. FIG. 4E corresponds to the 756 nm wavelength, and FIG. 4F corresponds to the 797 nm wavelength.

Here the absorption coefficient distributions shown in FIG. 4C and FIG. 4D are focused on. These absorption coefficient distributions were calculated with a 10° reconstruction view angle. The 10° reconstruction view angle means that the absorption coefficient can be calculated at high precision in a 127 mm×sin 10° about a 22 mm range from the center point of the curvature of the support. The absorber on the left side in each drawing is in this region, hence the shape thereof is clearly seen, just like FIG. 4A and FIG. 4B. The absorber on the right, on the other hand, is not within about a 22 mm radius range from the center point of the curvature of the support, hence a reconstruction artifact is generated in both FIG. 4C and FIG. 4D, where shapes are not accurately reproduced and contrast drops.

Thus the reconstruction artifact decreases and reproducibility of the shape improves if the reconstruction view angle widens.

In other words, in the second calculation method, it is preferable to use a wider reconstruction view angle. Then higher visibility can be implemented when the absorption coefficient distribution is imaged.

Now the oxygen saturation distribution will be described.

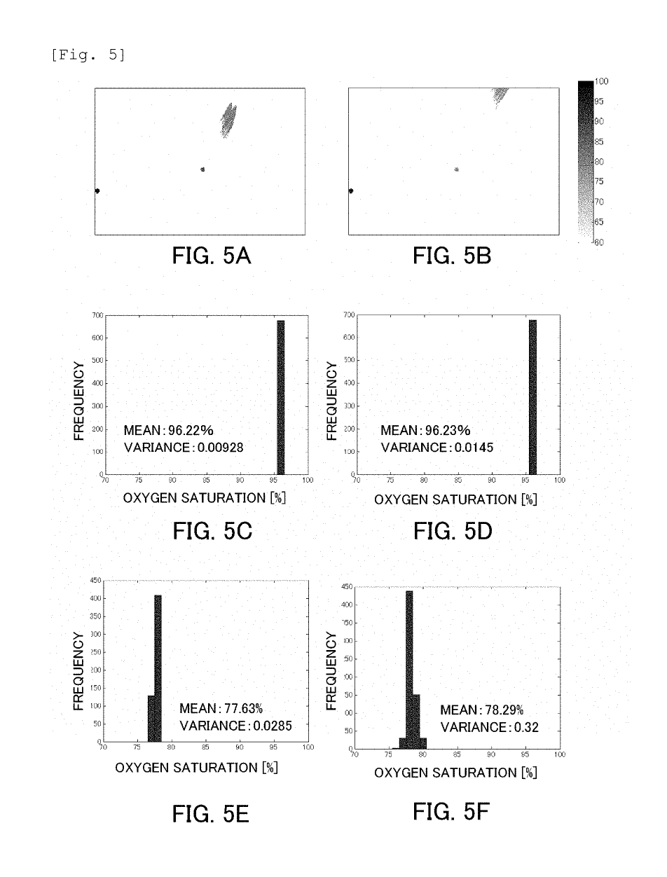

FIG. 5A is an oxygen saturation distribution calculated based on the absorption coefficient distributions shown in FIG. 4C and FIG. 4D, and FIG. 5B is an oxygen saturation distribution calculated based on the absorption coefficient distributions shown in FIG. 4E and FIG. 4F.

FIG. 5C is a histogram of the oxygen saturation corresponding to the absorber on the left side of FIG. 5A, and FIG. 5E is a histogram of the oxygen saturation corresponding to the absorber on the right side of FIG. 5A.

In the same manner, FIG. 5D is a histogram of the oxygen saturation corresponding to the absorber on the left side of FIG. 5B, and FIG. 5F is a histogram of the oxygen saturation corresponding to the absorber on the right side of FIG. 5B.

First a case of FIG. 5A (the reconstruction view angle is 10°) will be considered. As mentioned above, the true value of the oxygen saturation of the absorber on the left side of the drawing is 96%, and the true value of the oxygen saturation of the absorber on the right side is 76%.

According to the result in FIG. 5C, the mean is 96.21% and the variance is 0.00926%, and according to the result in FIG. 5E, the mean is 77.63% and the variance is 0.0285%.

Comparing these results with FIG. 5D and FIG. 5F, the oxygen saturation is closer to the true value, and the variance is smaller in FIG. 5C and FIG. 5E (the reconstruction view angle is 10°). This is because decreasing the reconstruction view angle makes it less likely to receive the influence of a streak artifact of other absorbers, and decreases errors in oxygen saturation.

In other words, in the first calculation method, it is preferable to use a much smaller reconstruction view angle. Thereby a more accurate result can be acquired when the oxygen saturation distribution is imaged.

Integrating the above results, it is preferable that the reconstruction view angle used for the second calculation method is larger than the reconstruction view angle used for the first calculation method. Thereby both the accuracy of the vascular imaging and the calculation accuracy of the oxygen saturation can be implemented.

### EXAMPLE 2

Example 2 is an example when the first calculation method includes averaging processing to reduce white noise. The configuration of the photoacoustic measurement apparatus according to Example 2 is the same as Example 1, except for the aspect to be described herein below.

As the light source 11, the photoacoustic measurement apparatus according to Example 2 uses an alexandrite laser which can emit 100 nanoseconds or shorter pulsed light at two wavelengths: 756 nm and 797 nm. As the optical system 12, a combination of a spatial propagation arm, a mirror, a lens and a diffusion plate is used.

In Example 2, the moving unit 43 is a unit to shift the support 41 in the X-Z direction.

In the photoacoustic measurement apparatus according to Example 2, the first optical characteristic acquisition unit 61 calculates the absorption coefficient distribution by calculating the initial sound pressure distribution using the UBP method, and dividing the light quantity distribution by the initial sound pressure distribution. Then in the calculated absorption coefficient distribution, an arithmetic mean is determined among adjacent voxels.

Further, the second optical characteristic acquisition unit 62 calculates the absorption coefficient distribution by calculating the initial sound pressure distribution using the UBP method, and dividing the light quantity distribution by the initial sound pressure distribution, just like the first absorption coefficient distribution calculation mechanism. The above mentioned arithmetic means, however, is not determined here.

Thus in Example 2, the processing to determine the arithmetic mean among peripheral voxels is included only in the first calculation method, and is not included in the second calculation method.

The effect when the first calculation method and the second calculation method are designed in this way will be described.

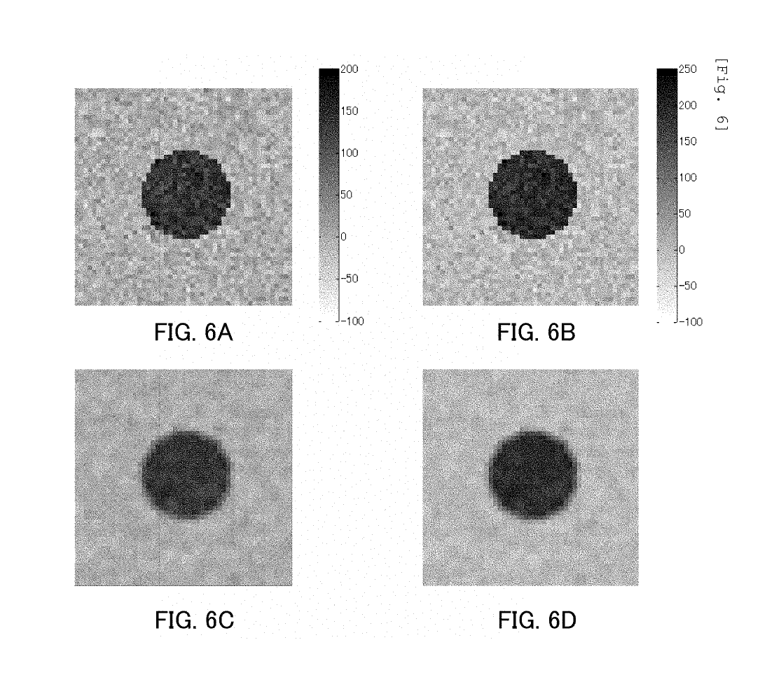

FIG. 6A is an absorption coefficient distribution acquired by irradiating the pulsed light having a 756 nm wavelength, and FIG. 6B is an absorption coefficient distribution acquired by irradiating the pulsed light having a 797 nm wavelength.

Each absorption coefficient distribution is an absorption coefficient distribution corresponding to that of a Φ2 mm artery, of which absorption coefficient is 0.138/mm or 0.189/mm, and oxygen saturation is 96%.

In the absorption coefficient distributions, white noise having a normal distribution of which variance is 20 is assumed to be a virtual absorption coefficient. The size of each drawing shown in FIG. 6A to FIG. 6D is 5 mm (Y direction)×5 mm (X direction), and the voxel size is 0.1 mm.

FIG. 6C is a result when the arithmetic means is determined using voxels located above, below, left and right in the absorption coefficient distribution shown in FIG. 6A. FIG. 6D is a result when the arithmetic means is determined using voxels located above, below, left and right in the absorption coefficient distribution shown in FIG. 6B. By determining the arithmetic means using the peripheral voxels, the edge of the light absorber becomes blurred, and the roughness caused by noise decreases.

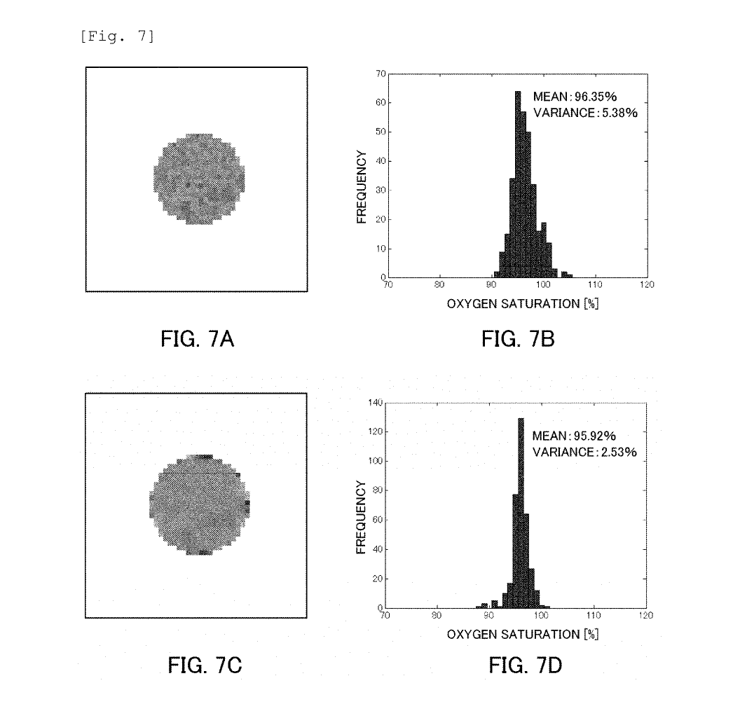

FIG. 7A to FIG. 7D are oxygen saturation distributions calculated using the absorption coefficient distributions shown in FIG. 6A to FIG. 6D and histograms thereof. FIG. 7A is the oxygen saturation distribution calculated using the absorption coefficient distributions shown in FIG. 6A and FIG. 6B, and FIG. 7B is the corresponding histogram.

FIG. 7C is the oxygen saturation distribution calculated using the absorption coefficient distribution shown in FIG. 6C and FIG. 6D, and FIG. 7D is the corresponding histogram.

When the arithmetic mean processing is performed on the absorption coefficient distributions, the calculated mean of the oxygen saturation is 95.92% and the variance is 2.53%. When the arithmetic means processing is not performed, the calculated mean of the oxygen saturation is 96.35% and the variance is 5.38%. In other words, accuracy of the oxygen saturation improves if the arithmetic means processing is performed on the absorption coefficient distribution.

However, when the arithmetic means processing is performed, the edge becomes blurred and accuracy of vascular imaging drops. Therefore it is preferable that the processing to determine the arithmetic means of peripheral voxels is included in the first calculation method, and is not included in the second calculation method. Thereby, both the accuracy of the vascular imaging and the calculation accuracy of the oxygen saturation can be implemented.

### EXAMPLE 3

Example 3 is an example when the second calculation method includes signal processing to delete an artifact, which is formed inside the object by multiple reflections of the acoustic wave.

A photoacoustic measurement apparatus according to Example 3 is an example corresponding to Embodiment 1. In other words, measurement is performed by compressing and holding an object using flat holding plates, and via the holding plates, a pulsed light is irradiated and an acoustic wave is acquired using a two-dimensional probe.

When the object is held by the flat holding plates, the multiple reflection of the acoustic wave is generated on the contacting surface between the object and the holding plate, or between the holding plate and the acoustic wave receiver, because of the difference of acoustic impedance, and thereby an artifact is generated.

Therefore in Example 3, the second optical characteristic acquisition unit 62 performs signal processing to delete the artifact, which is generated inside the object by the multiple reflection of the acoustic wave. The first optical characteristic acquisition unit 61, on the other hand, does not perform such signal processing.

Thus in Example 3, the processing to delete the artifact is included only in the second calculation method, and is not included in the first calculation method.

In this example, it is assumed that the photoacoustic signal acquired by the two-dimensional probe is signals that are three-dimensionally arrayed (arrayed signals) in a space having X, Y and Z axes (XY is the scanning plane, and Z is the time axis). A signal generated from a plane which is parallel with the two-dimensional probe, or a signal generated by this signal that is reflected by the plane which is parallel with the two-dimensional probe, becomes a signal which has low frequency components including DC, on the XY plane corresponding to a point in time of an arrayed signal.

Therefore these plane wave artifacts can be deleted by using a filter (high pass filter) to delete low frequency components, including DC components, in the XY direction, and as a result, the vascular imaging accuracy can be improved. This method is disclosed in Patent Literature 1.

This processing, however, may delete the original signal since not only the DC components but also low frequency components are deleted. If the original signal is deleted, the absorption coefficient ratio between wavelengths may be changed.

Therefore in Example 3, the first optical characteristic acquisition unit 61 calculates the absorption coefficient distribution without executing this plane wave artifact delete processing, and the second optical characteristic acquisition unit 62 executes the plane wave artifact delete processing first, and then calculates the absorption coefficient distribution.

Thus in Example 3, the processing to delete the plane wave artifact is included only in the second calculation method, and is not included in the first calculation method. Thereby both the accuracy of the vascular imaging and the calculation accuracy of the oxygen saturation can be implemented.

### EXAMPLE 4

Example 4 is an example when the second optical characteristic acquisition unit generates the absorption coefficient distribution by executing optimization processing, such as signal impulse response correction processing, blind deconvolution processing and spatial impulse response correction processing, or executing reconstruction processing, such as model base reconstruction method.

As disclosed in Patent Literature 2 and Patent Literature 3, these methods optimize the initial sound pressure distribution and the absorption coefficient distribution so that the value of a certain objective function is minimized. Depending on the objective function to be used, such an effect as improving visibility (e.g. improving resolution) and improving quantitativity can be demonstrated. On the other hand, these processing operations, which are executed for each wavelength, may change the absorption coefficient ratio between wavelengths. This is because the minimum point of the objective function cannot be uniquely determined, and the final position of the minimum point changes depending on the initial value.

Therefore in Example 4, the first optical characteristic acquisition unit 61 calculates the absorption coefficient distribution without executing such optimization processing, and the second optical characteristic acquisition unit 62 executes the optimization processing first, and then calculates the absorption coefficient distribution.

The optimization processing may be additional processing of the signal impulse response correction, the blind deconvolution, the spatial impulse response correction or the like, or may be a reconstruction method itself, such as model-based reconstruction.

Thus in Example 4, the optimization processing is included only in the second calculation method, and is not included in the first calculation method. Thereby both the accuracy of the vascular imaging and the calculation accuracy of the oxygen saturation can be implemented.

### EXAMPLE 5

Example 5 is an example when the second optical characteristic acquisition unit executes image processing to highlight the shape of a linear object (hereafter called “line highlighting processing”). By the line highlighting processing, the vascular portion can be highlighted.

The line enhancement processing is filter processing to extract linear objects from the image and highlight the objects. By this image processing, linear objects, such as blood vessels, can be clearly recognized. On the other hand, this processing, which is performed for each wavelength, may change the ratio of the absorption coefficients between wavelengths.

Therefore in Example 5, only the second optical characteristic acquisition unit 62 performs the line highlighting processing on the calculated absorption coefficient distribution, and the first optical characteristic acquisition unit 61 calculates the absorption coefficient distribution by a normal method.

Thus in Example 5, the line highlighting processing is included only in the second calculation method, and is not included in the first calculation method. Thereby both the accuracy of the vascular imaging and the calculation accuracy of the oxygen saturation can be implemented.

### (Modifications)

The description on each embodiment and description on the examples merely illustrate the present invention, and the present invention can be carried out by appropriately changing or combining the above embodiments and examples within a scope that does not depart from the spirit of the invention.

For example, the present invention may be carried out as a photoacoustic apparatus that executes at least a part of the above processing. The present invention may be carried out as a processing method including at least a part of the above processing executed by a photoacoustic apparatus. The above processing and units can be freely combined as long as technical inconsistencies are not generated.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transistory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD) (TM)), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-209193, filed on Oct. 10, 2014, which is hereby incorporated by reference herein in its entirety.

### REFERENCE SIGNS LIST

1 light irradiation unit
4 acoustic wave receiver
5 signal processor
6 calculation processor

1. A photoacoustic apparatus, comprising:
a light source configured to irradiate an object with a plurality of pulses of pulsed light, the pulsed light having a plurality of different wavelengths;
an acoustic wave receiver configured to receive an acoustic wave generated from the object, to which the pulsed light has been irradiated, and convert the acoustic wave into an electric signal;
a first information acquisition unit configured to acquire a first absorption coefficient distribution inside the object, on the basis of the electric signal, using a first calculation method;
a second information acquisition unit configured to acquire a second absorption coefficient distribution inside the object, on the basis of the electric signal, using a second calculation method;
a third information acquisition unit configured to calculate the distribution of functional information on the interior of the object on the basis of the plurality of first absorption coefficient distributions acquired by irradiating the plurality of pulsed light having different wavelengths respectively; and
an image generation unit configured to generate an image by correcting the distribution of the functional information on the basis of the second absorption coefficient distribution,
wherein the second calculation method is a method that can implement higher visibility than the first calculation method when the absorption coefficient distribution is imaged.

2. The photoacoustic apparatus according to claim 1, wherein the second calculation method includes processing to decrease artifact, or processing to improve resolution.

3. The photoacoustic apparatus according to claim 1, wherein the second calculation method includes processing to perform impulse response correction on the electric signal.

4. The photoacoustic apparatus according to claim 1, wherein the second calculation method includes processing to perform spatial impulse response correction.

5. The photoacoustic apparatus according to claim 1, wherein the second calculation method includes blind deconvolution processing.

6. The photoacoustic apparatus according to claim 1, wherein a reconstruction view angle to calculate an absorption coefficient in the second calculation method is larger than that in the first calculation method.

7. The photoacoustic apparatus according to claim 1, wherein the second calculation method is for calculating an absorption coefficient by a model-based reconstruction method.

8. The photoacoustic apparatus according to claim 1, wherein the first calculation method is a method that can calculate a ratio of absorption coefficients between wavelengths at higher accuracy than the second calculation method.

9. The photoacoustic apparatus according to claim 1, wherein the reconstruction view angle to calculate the absorption coefficient in the first calculation method is smaller than that in the second calculation method.

10. The photoacoustic apparatus according to claim 1, wherein the first calculation method includes processing to determine an arithmetic mean among peripheral pixels or voxels.

11. The photoacoustic apparatus according to claim 1, wherein the image generation unit generates an image in which values indicated in the distribution of the functional information are expressed as hue and values indicated in the second absorption coefficient distribution are expressed as lightness.

12. The photoacoustic apparatus according to claim 1, wherein the third information acquisition unit acquires oxygen saturation distribution as the distribution of the functional information.

13. A processing method for a photoacoustic apparatus having a light source configured to irradiate an object with a plurality of pulses of pulsed light the pulsed light having a plurality of different wavelengths, and an acoustic wave receiver configured to receive an acoustic wave generated from the object, to which the pulsed light has been irradiated, and convert the acoustic wave into an electric signal, the method comprising:
a first information acquisition step of acquiring a first absorption coefficient distribution inside the object, on the basis of the electric signal, using a first calculation method;
a second information acquisition step of acquiring a second absorption coefficient distribution inside the object, on the basis of the electric signal, using a second calculation method;
a third information acquisition step of calculating a distribution of functional information on the interior of the object on the basis of the plurality of first absorption coefficient distributions acquired by irradiating the plurality of pulsed light having different wavelengths respectively; and
an image generation step of generating an image by correcting the distribution of the functional information on the basis of the second absorption coefficient distribution,
wherein the second calculation method is a method that can implement higher visibility than the first calculation method when the absorption coefficient distribution is imaged.
